(19) United States
(12) Patent Application Publication
(10) Pub. No.: US 2005/0240251 A1
(43) Pub. Date: Oct. 27, 2005

(54) Apparatus and method for applying cooling substances to pressure points in the human body

(76) Inventor: Deborah A. Smith, West Orange, NJ (US)

Correspondence Address: RICHARD L. BIGELOW, 203 TREMONT STREET, NEWINGTON, CT, 06111, US

(21) Appl. No.: 11/102,361

(22) Filed: Apr. 9, 2005

Related U.S. Application Data
(60) Provisional application No. 60/564,855, filed on Apr. 23, 2004.

(51) Int. Cl. A61F 7/00

(52) U.S. Cl. 607/114

(57) ABSTRACT

A mechanism and method is presented for applying a cool object to various pressure points of the body to relieve the effects of hot flashes. In particular, the present invention involves cooling small internal sacks in a freezer, removing them when a hot flash occurs, placing the sacks in larger external pouches and then attaching the pouches to either a bracelet or watch around the wrist or a necklace such that the cool pouches can provide relief to the pressure points on the inside of the wrist, ankles or the back of the neck. Application of the cold pouches to these pressure points provides relief from the effects of hot flashes.

### CROSS REFERENCE TO RELATED APPLICATIONS

This non-provisional patent application claims priority based on provisional patent application 60/564,855 dated Apr. 23, 2004.

### FEDERAL RESEARCH STATEMENT

None

### BACKGROUND OF THE INVENTION

This invention relates to an apparatus and method for the application of cooling substances to pressure points in the human body. More specifically, the invention relates to the application of cool or ice-cold materials to the specific pressure points of the back of the neck, the wrists, and the ankles in order to alleviate the discomfort of hot flashes suffered by women in various stages of menopause. Hot flashes can occur at any time and at any place and are a source of considerable discomfort and consternation to many menopausal women. In the past, many women have resorted to using various forms of medication to lessen the effects of menopause and attendant hot flashes. In light of recent research that has indicated that deleterious physiological effects may accompany the use of aforesaid medications, more and more women have adopted a more natural approach to combat menopause and the attendant hot flashes. Consistent with the more natural approaches, some women have found that applying cool or cold materials to various pressure points in the body, such as the back of the neck and the wrists, provides considerable and immediate relief to the discomfort of hot flashes.

Since hot flashes can occur at any time and at any place, including work, school, various social settings, as well as in the comfort of one's home, the problem for women has been to find a socially acceptable apparatus and means for applying and maintaining the cold material to the pressure points. The present inventions solve that problem by providing stylish devices that contain cold material that can be applied and maintained to important pressure points on the back of the neck, the wrists, and the ankles for a significant period of time in order to provide rapid and continuing relief.

### SUMMARY OF THE INVENTION

The present inventions are directed toward devices that can be cooled and worn on various pressure points in the body, including the back of the neck, the wrists and the ankles, in order to effectively combat the effects of hot flashes.

Hot flashes are associated with hormonal changes in menopausal women. The hormonal imbalances generate excess heat that the body must disperse. Much of the excess heat is dispersed from the head and the neck. Some is also dispersed through other parts of the upper body, including the arms.

Specifically, the device that is designed to cool the pressure point on the back of the neck (the “neck pouch”) consists of a small external pouch (between 2″ and 3″ wide and between 2″ and 3″ long and between 0.4″ and 0.75″ deep). The external pouch is made of an aesthetically pleasing material that also has a fairly high thermal conductivity, such as a shimmering gauze-like fabric, and is so designed that it may be re-sealable with a device such as seen on a Zip Lock bag or with Velcro. The neck pouch is further designed such that it can be easily attached to the back of a necklace so that it may be easily and quickly applied and removed.

The device that is designed to cool the pressure point on the wrist (the “wrist pouch”) is similar to the neck pouch except that it is a square between 1″ and 2″ on a side and between 0.4″ and 0.75″ deep. The wrist pouch is also made of an aesthetically pleasing material, such as a shimmering gauze-like fabric. It can also be resealable. The wrist pouch is designed to be attached to a watch or bracelet. Generally, the wrist pouch will be placed against the underside of the wrist.

The wrist pouch can also be used to cool pressure points around the ankles using an ankle bracelet. The ankle bracelet can hold the pouch in place around the ankle or lower leg in much the same way that a wrist bracelet holds the wrist pouch in place.

The key to the effectiveness of the neck and wrist pouches is the cooling mechanism. The cooling mechanism consists of a small internal sack that is designed to fit inside the external neck or wrist pouch. Inside the internal sack are small objects, such as polyester or metal beads or a hard organic substance such as rice. The internal sack can be cooled in a freezer for a significant period of time such that it attains a temperature approximating that of the freezer air. When a woman begins to experience a “hot flash”, she can remove one of the frozen internal sacks, insert it in the neck or wrist pouch and, within a very short period of time, begin to experience a soothing cooling sensation. The relief can be almost immediate, thereby rendering it superior to the use of medications which often takes a significant amount of time to be effective.

The internal sack for the neck pouch is designed to fit inside the neck pouch and is generally configured to be 1.8″ to 2.8″ long by 1.8 to 2.8″ wide by less than 0.5″ deep. The internal sack for the wrist pouch is similarly designed to fit inside the wrist pouch and is generally configured to be 0.75″ to 1.75″ long by 0.75″ to 1.75″ wide by less than 0.5″ deep.

### BRIEF DESCRIPTION OF THE DRAWINGS

FIG. 1 shows the internal sack as it is inserted into the external neck pouch.

FIG. 2 shows the internal sack as it is inserted into the external wrist pouch.

FIG. 3 shows the relative position of the external pouch containing the cold internal sack on the back of the neck.

FIG. 4 shows the wrist pouch attached to a watch band and positioned on the wrist.

### DETAILED DESCRIPTION OF THE INVENTION

The present invention is directed toward devices that can be cooled and worn on various pressure points in the body, including the back of the neck, the wrists and the ankles, in order to effectively combat the effects of hot flashes. Specifically, the inventions comprise internal sacks that can be cooled in a refrigerator or freezer and placed into external pouches that can be worn on various pressure points on the body such as the back of the neck, the wrists, or the ankles in order to cool said pressure points and provide relief from hot flashes.

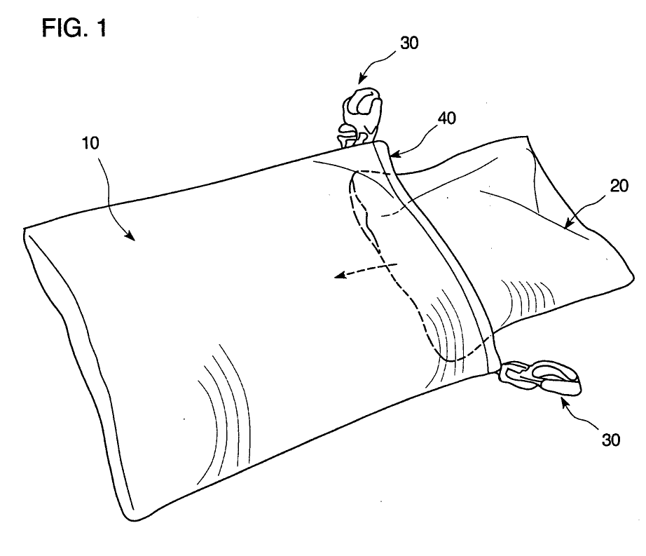

Referring to the figures and in particular to FIG. 1, the external neck pouch 10 and internal sack 20 are shown. The neck pouch 10 is closed on three ends and is open at the top 40. FIG. 1 shows the manner in which the internal sack is placed into the external pouch. FIG. 1 also shows clasps 30 that are attached to the top of the external neck pouch. The internal sack 20 is closed on all sides and is filled with a material or substance that is capable of remaining cool for a significant period of time. The preferred means of utilizing the present invention is for the internal sack 20 to be placed in a freezer for a period of time sufficient to cool it. When a hot flash occurs, the internal sack 20 is removed from the freezer, inserted into the external pouch 10, attached to a necklace and placed on the back of the neck. Alternatively, in the event the internal sack 20 is not available or has not been cooled a sufficient period of time, ice can be placed into a sealable plastic bag and placed into the external pouch 10.

The external pouch 10 and internal sack 20 are made of a sturdy fabric that can also be stylish. In the preferred embodiment, the pouch 10 and sack 20 are made of a shimmering gauze fabric. The preferred material that is placed in the internal sack 20 is rice.

The external neck pouch 10 is designed to be between 2″ and 4″ long by between 2″ and 3″ wide and 0.4″ to 0.75″ deep. The internal sack 20 is designed to fit inside the neck pouch and is generally configured to be 1.8″ to 2.8″ long by 1.8 to 2.8″ wide by less than 0.5″ deep.

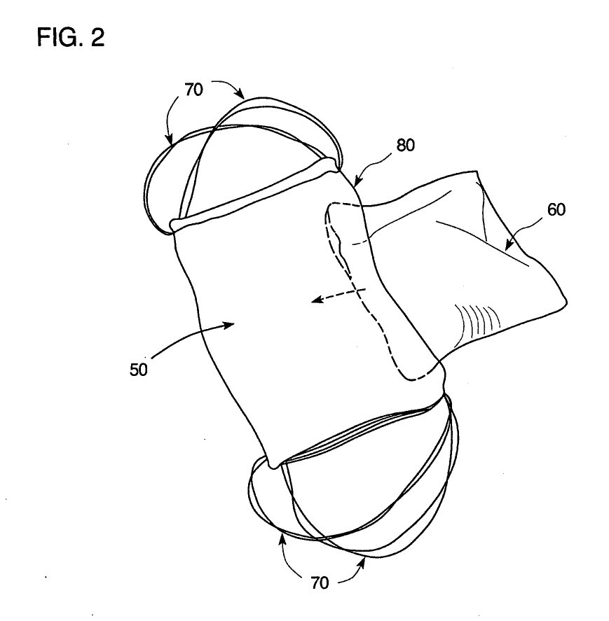

Referring to FIG. 2, the external wrist pouch 50 and internal sack 60 are shown. The wrist pouch 50 is closed on three ends and is open at the top 80. FIG. 2 shows the manner in which the internal sack is placed into the external pouch. FIG. 2 also shows loops 70 that are attached to the ends of the external wrist pouch. The internal sack 60 is closed on all sides and is filled with a material or substance that can be frozen. The external pouch 50 and internal sack 60 are made of a sturdy fabric that can also be stylish. In the preferred embodiment, the pouch 50 and sack 60 are made of a shimmering gauze fabric. The preferred material that is placed in the internal sack 60 is rice.

The external wrist pouch 50 is designed to be a square between 1″ and 2″ on a side and between 0.4″ and 0.75″ deep. The internal sack 60 is designed to fit inside the wrist pouch 50 and is to be 0.75″ to 1.75″ long by 0.75″ to 1.75″ wide by less than 0.5″ deep. The external wrist pouch 50 and internal sack 60 are generally smaller than the external neck pouch 10 and internal sack 20 shown in FIG. 1.

In a similar manner, the wrist pouch can be used with an ankle bracelet to apply cooling substances to pressure points in the ankles or lower leg.

The preferred means of utilizing the present invention is for the internal sack 60 to be placed in a freezer for a period of time sufficient to cool it. When a hot flash occurs, the internal sack 60 is removed from the freezer, inserted into the external pouch 50, attached to a watch band or bracelet and placed on the wrist. Alternatively, in the event the internal sack 60 is not available or has not been cooled a sufficient period of time, ice can be placed into a sealable plastic bag and placed into the external pouch 50.

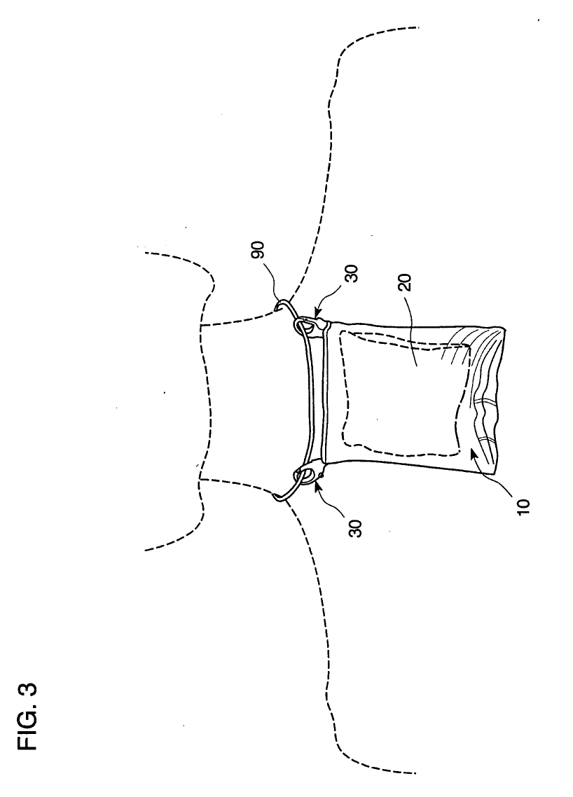

Referring to FIG. 3, the external neck pouch 10 containing the cold internal sack 20 is shown as worn against the back of the neck. The pouch is designed to be attached to a necklace 90 by means of passing the necklace 90 through at least two clasps 30.

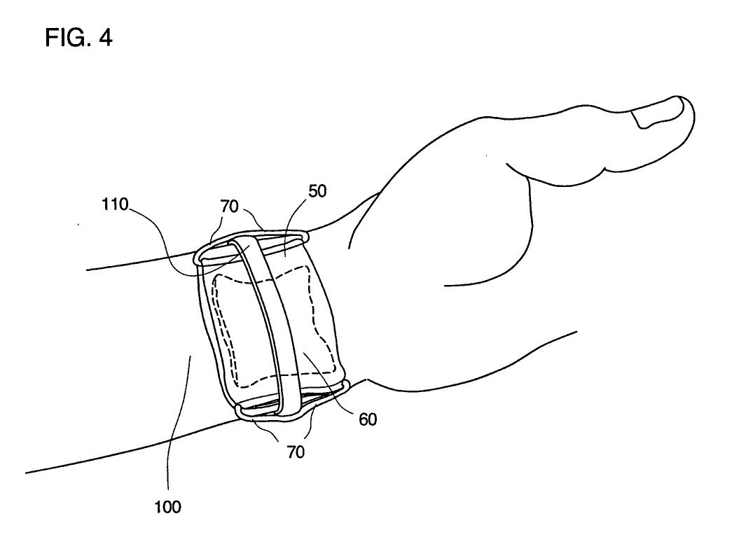

Referring to FIG. 4, the external wrist pouch 50 containing the cold internal sack 60 is shown as worn on the inner part of the wrist 100. The external wrist pouch 50 is designed to be attached to a bracelet or watch 110 by running the bracelet or watch 110 through the loops 70 on the sides of the pouch 50. In the preferred mode, the pouch 50 is held against the wrist 100 by running the bracelet or watch 110 on the outside of the pouch 50.

1. An apparatus for applying cooling substances to pressure points of the body comprising:
a. an internal sack that contains a coolable material,
b. an external sack that is open on at least one side such that the internal sack can be placed into the external sack, and
c. a method whereby the external sack can be applied to a pressure point of the body.

2. An apparatus as in claim 1 wherein the coolable material contained in the internal sack is capable of being placed into a refrigerator or freezer in order to cool said material,

3. An apparatus as in claim 1 wherein the internal sack is between 1.5 and 2.5 inches on a side.

4. An apparatus as in claim 1 wherein the external sack is between 2″ and 3″ wide and between 2″ and 3″ long and between 0.4″ and 0.75″ deep.

5. An apparatus as in claim 1, 2, 3 or 4 wherein at least two connecting devices are fixedly attached to the external sack in such a manner that that the external sack can be attached to a necklace or other such device such that the external sack can be optimally positioned to lie against and cool pressure points on the back of the neck.

6. An apparatus for applying cooling substances to pressure points of the body comprising:
a. an internal sack that contains a coolable material,
b. an external sack that is open on at least one side such that the internal sack can be placed into the external sack, and
c. a method whereby the external sack can be applied to a pressure point of the body.

7. An apparatus as in claim 6 wherein the coolable material contained in the internal sack is capable of being placed into a refrigerator or freezer in order to cool said material,

8. An apparatus as in claim 6 wherein the internal sack is between 0.75 and 1.7.5 inches on a side.

9. An apparatus as in claim 6 wherein the external sack is between 1″ and 2″ wide and between 1″ and 2″ long and between 0.4″ and 0.75″ deep.

10. An apparatus as in claim 6, 7, 8 or 9 wherein at least two connecting devices are fixedly attached to the external sack in such a manner that that the external sack can be attached to or held in place by a wrist or ankle bracelet, watch or other such device.
